(19) United States
(12) Patent Application Publication
(10) Pub. No.: US 2013/0035557 A1
(43) Pub. Date: Feb. 7, 2013

(54) METHOD AND APPARATUS FOR LEVELING PRESSURE TRANSDUCERS

(71) Applicants: Joshua J. Leppert, Coeur D Alene, ID (US); Joseph C. Livengood, Fort Collins, CO (US); Barry T. Phillips, Fort Collins, CO (US)

(72) Inventors: Joshua J. Leppert, Coeur D Alene, ID (US); Joseph C. Livengood, Fort Collins, CO (US); Barry T. Phillips, Fort Collins, CO (US)

(73) Assignee: LEPPERT LEVEL LLC, Coeur D Alene, ID (US)

(21) Appl. No.: 13/565,631

(22) Filed: Aug. 2, 2012

Related U.S. Application Data
(60) Provisional application No. 61/514,128, filed on Aug. 2, 2011.

(51) Int. Cl. G01L 19/02; A61B 5/03; A61B 5/0215; G01C 9/02; G01C 9/24

(52) U.S. Cl. 600/300; 33/374; 33/301; 73/700

(57) ABSTRACT

A hinged level and method for utilizing the same in connection with the leveling of a pressure transducer with respect to a reference point are provided. The hinged level includes a plurality of elongate members interconnected to one another by a hinge. The hinge allows the elongate members to be pivoted relative to one another within a first plane. A level device is aligned with the first plane. The level or elevation of a pressure transducer relative to a reference point, which corresponds to the elevation of an implanted catheter, is determined by placing the free ends of the hinged level relative to the reference point and the pressure transducer. The height of the pressure transducer is then adjusted until the level device indicates that the elongate members are level.

### CROSS-REFERENCE TO RELATED APPLICATIONS

This application claims the benefit of U.S. Provisional Patent Application Ser. No. 61/514,128, filed Aug. 2, 2011, the entire disclosure of which is hereby incorporated herein by reference.

### FIELD

Methods and apparatuses for leveling pressure transducers are provided.

### BACKGROUND

Information regarding the pressure of fluid within a body is useful in assessing the health of individuals. In order to obtain a pressure reading a catheter, such as an arterial or intracranial catheter, is implanted in the patient. The catheter is interconnected to a pressure transducer or manometer external to the patient by a tube containing a pressure transmitting fluid. In order to obtain an accurate reading of the internal pressure to be measured, the level or elevation of the pressure transducer must be the same as an anatomic reference point.

In order to place the pressure transducer at the same level as the anatomic reference point, a straight dowel, with or without a spirit level, or a conventional carpenter level, can be used. However, the use of a straight dowel or level is flawed because there often is no straight path between the reference point on the patient corresponding to the level of the anatomic reference point and the transducer. Other systems have been developed that include the use of cords carrying a bubble level. However, such systems do not remove the need for a straight path between the reference point on the body and the pressure transducer. Still other systems have associated leveling tubes and electronic sensors, or systems that involve the use of radio frequency opaque materials and radio frequency sensors, with catheters, in order to determine the elevation of the anatomic reference point and/or the catheter. However, these systems are relatively complex and require that measuring or reference components be placed in the patient.

Accordingly, it would be desirable to provide a relatively simple, reliable, and inexpensive device for use in placing a pressure transducer at the level of an anatomic reference point that was capable of operation even when a straight path between the reference point on the patient and the pressure transducer is obstructed.

### SUMMARY

The present invention is directed to solving these and other problems and disadvantages of the prior art. Embodiments of the present invention provide a method and apparatus for leveling pressure transducers that incorporates a hinged level. More particularly, a leveling device is disclosed that includes two rigid, generally planar elongate members or arms joined to one another by a hinge that allows an angle between the two elongate members to be varied. In addition, the hinged level includes a level device.

In accordance with embodiments of the present invention, a first one of the elongate members is interconnected to a second one of the elongate members such that the two members can be placed at any angle with respect to one another within a first plane. The level device is parallel to the first plane. Accordingly, the level device indicates that a free end of the first member is at the same elevation as the free end of the second member when the level device is level. In at least some embodiments the level device may comprise a bubble level.

Methods in accordance with embodiments of the present invention include providing two rigid members that are interconnected to one another by a hinge that permits an angle between the elongate members to be varied within a first plane, and that includes a level device that is parallel to the first plane. Placement of a pressure transducer at the same level or elevation as a reference point on a body, corresponding to the location of an implanted or internal catheter, is accomplished by placing a first, free end of the first member at or proximate to the reference point. The method further includes placing a free end of the second elongate member at or proximate to the elevation or level of the pressure transducer. Where the horizontal distance between the reference point on the patient and the pressure transducer is less than the fully extended length of the hinged level device, the elongate members can be pivoted at the hinge, in order to place the free ends of the elongate members at the required distance from one another. In addition, the resulting angle between the elongate members can be used to advantage, for example to avoid equipment, such as IV poles or bed frames, that lie between the reference point on the patient and the pressure transducer. If the level device indicates that the hinged level device is tilted when the free ends are at the reference point and the pressure transducer respectively, the level or elevation of the pressure transducer can be changed, until the bubble within the level device is centered. When the bubble of the level device is centered with the free end of the first elongate member at the reference point on the body and the free end of the second elongate member at the level of the pressure transducer, the pressure transducer can be considered at the same level as the catheter within the body.

Additional features and advantages of embodiments of the present invention will become more readily apparent from the following description, particularly when taken together with the accompanying drawings.

### BRIEF DESCRIPTION OF THE DRAWINGS

FIG. 1 illustrates a hinged level in accordance with embodiments of the present invention in an operative environment;

FIG. 2A illustrates a hinged level in accordance with embodiments of the present invention in a top plan view, with the elongate members at a first exemplary angle with respect to one another;

FIG. 2B illustrates a hinged level in accordance with embodiments of the present invention in a top plan view with the elongate members at a second exemplary angle with respect to one another;

FIG. 2C illustrates a hinged level in accordance with embodiments of the present invention in a top plan view with the elongate members at a third exemplary angle with respect to one another;

FIG. 3 is a bottom plan view of a hinged level in accordance with embodiments of the present invention, with the elongate members at the first exemplary angle with respect to one another;

FIG. 4 is a view in elevation of a hinged level in accordance with embodiments of the present invention;

FIG. 5 is a top plan view of the first elongate member of a hinged level in accordance with embodiments of the present invention;

FIG. 6 is a top plan view of the second elongate member of a hinged level in accordance with embodiments of the present invention; and

FIG. 7 is a flowchart illustrating aspects of the use and operation of a hinged level in accordance with embodiments of the present invention.

### DETAILED DESCRIPTION

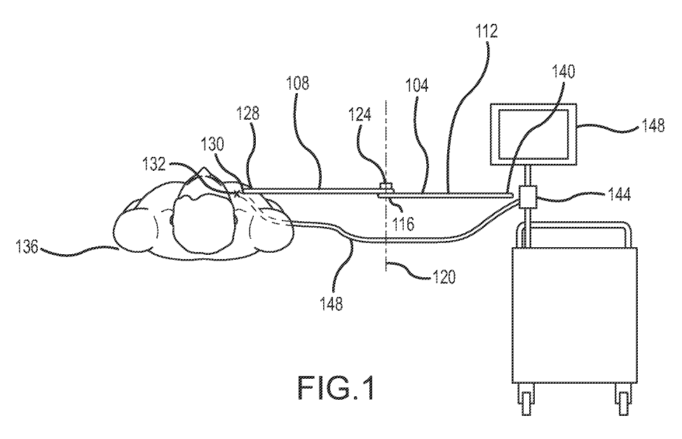

FIG. 1 illustrates a hinged level 104 in accordance with embodiments of the present invention, in an exemplary operating environment. More particularly, the hinged level 104 includes a first rigid arm or elongate member 108 interconnected to a second rigid arm or elongate member 112 by a hinge 116. In general, the hinge 116 allows the first elongate member 108 to be pivoted or rotated about a pivot axis 120 of the hinge 116 relative to the second elongate member 112. The hinged level 104 additionally includes a level device 124 that indicates whether a reference plane corresponding or parallel to a base or mounting surface of the level device 124 is level. In accordance with at least some embodiments of the present invention, the level device 124 is located at or near the pivot axis 120 of the hinge 116. The reference plane of the level device 124 lies in a plane that is in the same as and/or is parallel to the plane in which the elongate members 108 and 112 move when they are rotated about the pivot axis 120. Examples of a level device 124 that can be included as part of a hinged level 104 in accordance with embodiments of the present invention include, but are not limited to, a bubble level, a set of spirit levels, or an electronic level.

In use, a first or free end 128 of the first elongate member 108 is positioned proximate to and at the same level as a reference point 130. The reference point 130 can comprise an external anatomical feature or a point referenced to an anatomical feature that corresponds to the level in the body of the location from which an accurate pressure reading is desired. The first or free end 140 of the second elongate member 112 is placed at the level or elevation at which the level device 124 indicates that the elongate members 112 are level. As can be appreciated by one of skill in the art after consideration of the present disclosure, the free ends 128 and 140 of the elongate members 108 and 112 are at the same level or elevation when the level device 124 is itself level. A pressure transducer 144, which is interconnected to the catheter 132 by a tube 148 containing a pressure transmitting fluid, is then placed at the level of the free end 140 of the second elongate member 112. Once the pressure transducer 144 is at the level of the reference point 130, a signal from the pressure transducer 144, which for example can be displayed by a monitor 148, can be used for assessing the condition of the patient 136.

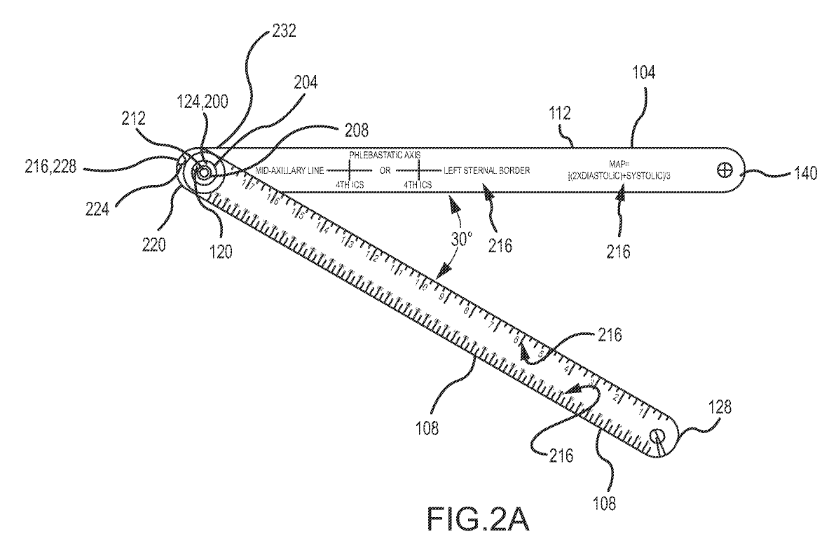

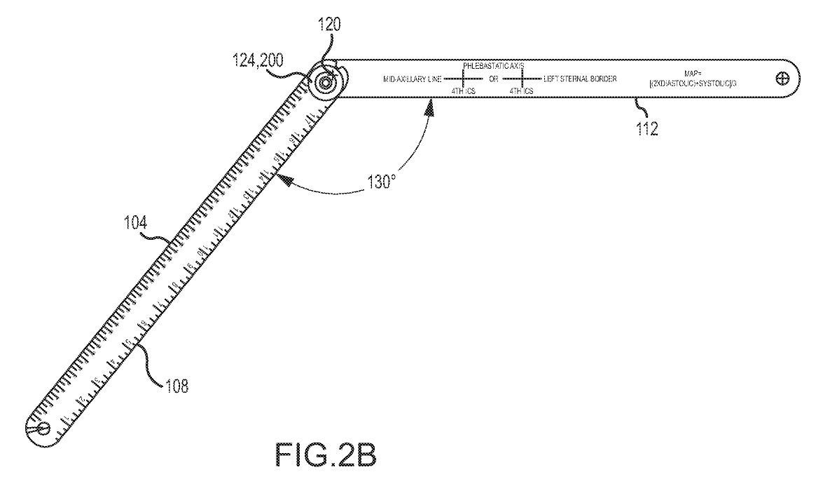

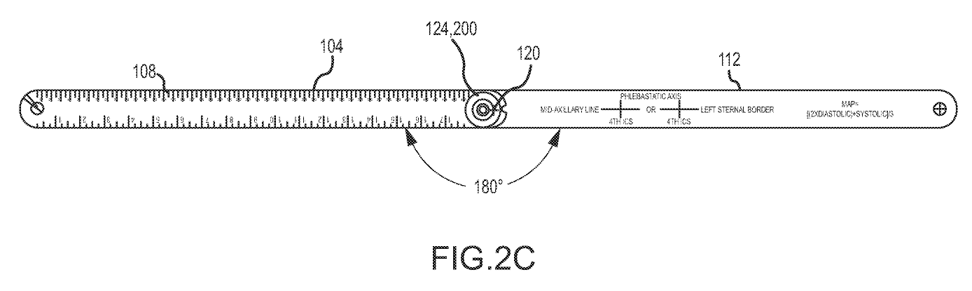

FIG. 2A is a top plan view of a hinged level 104 in accordance with embodiments of the present invention, with the first elongate member 108 at a first exemplary angle of about 30° with respect to the second elongate member 112. In this particular example, the level device 124 comprises a bubble level 200. As shown, a level device 124 comprising a bubble level 200 can include leveling marks 204 and 208, to help a user determine when the bubble 212 is centered, indicating that the hinged level 104 device is level.

In addition, the hinged level 104 can include various markings and other features. For instance, the first elongate member 108 can have markings in standard and/or metric units, to allow use of the hinged level 104 as a ruler. As another example, the second elongate member 112 can include reference information, including information that is helpful to a practitioner in locating a reference point on a patient or calculating values such as the mean arterial pressure. As yet another example, at a second end 220 of the first elongate member 108 opposite the first end 128, a notch 224 can be provided. More particularly, the notch 224 can function to reveal a marking 216 comprising an angular value 228 on the second end 232 of the second elongate member 112. This angular value 228 can indicate the angle between the first elongate member 108 and the second elongate member 112. Accordingly, the hinged level 104 can, in at least some embodiments, function as a protractor.

FIG. 2B is a top plan view of a hinged level 104 in accordance with embodiments of the present invention, with the first elongate member 108 at a second exemplary angle of about 130° with respect to the second elongate member 112. The hinged level 104 is shown with the first elongate member 104 at a third exemplary angle of about 180° with respect to the second elongate member 112 in FIG. 2C. In general, the hinged level 104 can be arranged such that the first 108 and second 112 elongate members are at any angle with respect to one another within a first plane that is perpendicular to the pivot axis 120 of the hinge 116. Moreover, the first plane contains and/or is parallel to the reference plane of the level device 124.

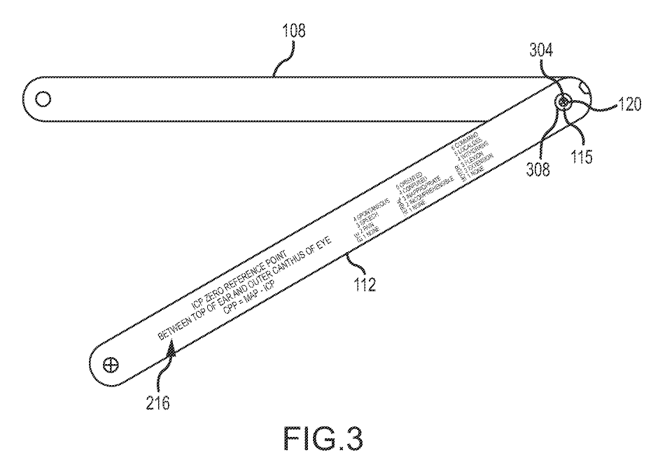

FIG. 3 is a bottom plan view of a hinged level in accordance with embodiments of the present invention, with the elongate members 108 and 112 at a first exemplary angle with respect to one another. As shown in the figure, the pivot axis 120 can correspond to a pivot member 304, such as a rivet or bolt that forms a part of the hinge 116 interconnecting the first elongate member 108 to the second elongate member 112. For example, the pivot member 304 may comprise a threaded member that is fixed to the first elongate member 108, extends through a hole in the second elongate member 112, to which a threaded fastener 308, such as a nut or thumbscrew, is connected to secure the first 108 and second 112 elongate members to one another. As other examples, the hinge 116 can comprise a swaged joint, a riveted joint, and/or a fastener that extends through holes in the elongate members 108 and 112. As also shown in the figure, additional indicia 216 can be formed on the bottom sides of the elongate members 108 and 112. For example, information concerning the location of the intracranial pressure zero reference point, a legend of patient eye, verbal, and motor status identifiers, or other information helpful to a practitioner can be included.

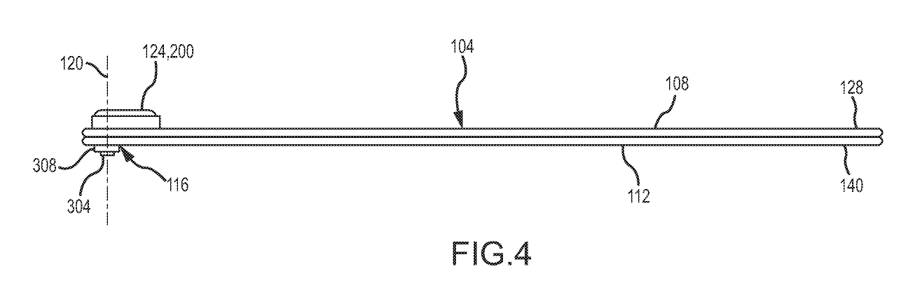

FIG. 4 is a view in elevation of a hinged level 104 in accordance with embodiments of the present invention. As shown in FIG. 4, the first 108 and second 112 elongate members are closely aligned in elevation, to provide a suitably accurate indication of whether a reference point on the patient 136 and the pressure transducer 144 are at the same elevation or level. A washer, for example a nylon washer, can be placed between the elongate members 108 and 112 about the pivot axis 120 to prevent binding. In addition, it can be seen that the first elongate member 108, the second elongate member 112, and the level device 124 are aligned such that they occupy parallel planes. Moreover, these parallel planes are sufficiently close to one another that the free ends 128 and 140 of the elongate members 108 and 112 are within 1.0 inch or less of one another in elevation when the hinged level 104 is level. In accordance with still other embodiments, the parallel planes are within 0.5 inch or less of one another. FIG. 4 also shows the pivot member 304 and the threaded fastener 308 of a hinge 116 in accordance with embodiments of the present invention. In accordance with further embodiments, one of the elongate members 108 or 112 may be longer than the other, and can include a step or transition beyond the end of the free end of the other elongate member so that at least the free ends 128 and 140 move in the same plane when elongate members 108 and 112 are moved about the pivot axis 120 to change the spacing between the free ends 128 and 140.

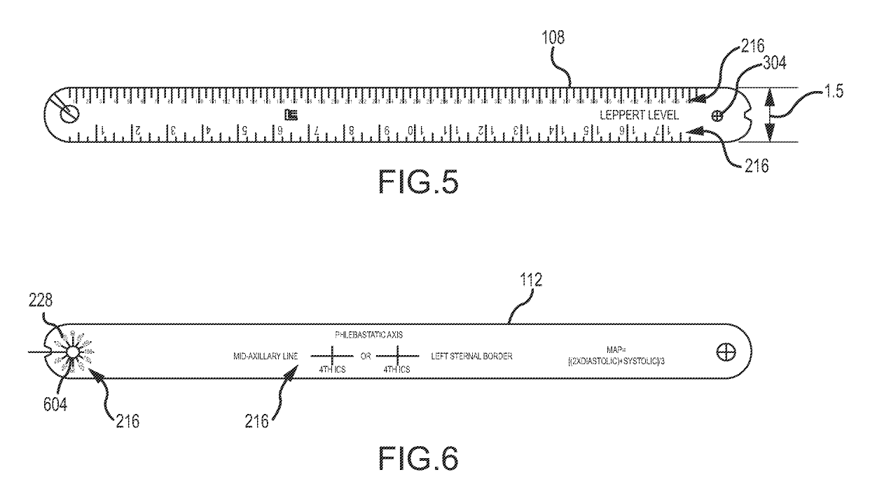

FIG. 5 is a top plan view of a first elongate member 108 in accordance with embodiments of the present invention. FIG. 6 is a top plan view of the second elongate member 112 of a hinged level 104 in accordance with embodiments of the present invention. Comparing FIGS. 5 and 6, it can be seen that the elongate members 108 and 112 may be generally identical to one another, except that the first elongate member 108 can include a pivot member 304, and the second elongate member 112 includes a hole 604 that receives the pivot member 304 (see FIG. 3) when the first elongate member 108 and the second elongate member 112 are interconnected to one another. In addition, any indicia included on the respective elongate members 108 and 112 can be different. For instance, while the first elongate member 108 may include indicia that facilitate use of the hinged level 104 as a device to measure distances, the second elongate member 112 can include indicia 216, 228 that allows the hinged level 104 to be used to measure angles.

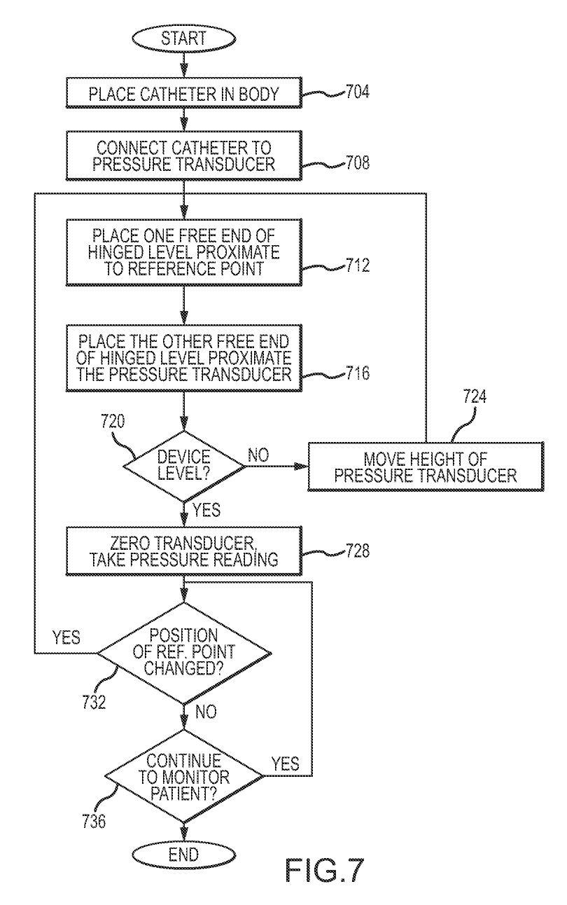

FIG. 7 is a flowchart illustrating aspects of the use and operation of a hinged level 104 in accordance with embodiments of the present invention. Initially, at step 704, a catheter 132 is placed at a desired location within the patient 136. The catheter 132 can then be interconnected to the pressure transducer 144 by the tube 148 containing the pressure transmitting fluid (step 708). At this point, in order to obtain an accurate reading of the pressure within the body of the patient 136, the pressure transducer 144 must be placed at the same level as a reference point 130.

The process of leveling the reference point 130 and the pressure transducer 144 can advantageously be performed using a hinged level 104 as described herein, using the following steps. In particular, a free end 128 or 140 of an elongate member 108 or 112 is placed proximate to the reference point 130 on the patient's body 136 (step 712). As can be appreciated by one of skill in the art, the reference point 130 can comprise a point that is itself referenced to an anatomical structure or feature of the patient 136. In accordance with still other embodiments, the reference point 130 can correspond to a point that is empirically determined to correspond to a location inside the body for which a pressure measurement is desired. Next, the other free end 128 or 140 of the first 108 or second 112 elongate members is placed proximate to the pressure transducer 144 (step 716). With each free end 128 and 140 aligned with one of the reference point and the pressure transducer 144, a determination is made as to whether the level device 124 indicates that the device 104 is level. For example, where the level device 124 is a bubble level 200, a determination is made as to whether the bubble 212 of the bubble level 200 is centered within the reference marks 204 and 208 (step 720). If the level device 124 indicates that the hinged level 104 device is not level, the height of the pressure transducer 144 is changed (step 724). The level device 124 is then again checked to determine whether it is level the free ends 128 and 140 are in position at the reference point 130 and the pressure transducer 144. Once the level device 124 indicates that the device is level, while the free ends 128 and 140 are proximate the reference point and the pressure transducer 144, the pressure transducer 144 is zeroed and a pressure reading can be taken (step 728). In general, a pressure reading can be taken at any time after the catheter 132 has been interconnected to the pressure transducer 144. However, the pressure reading provided by the pressure transducer 144 will not be accurate unless the pressure transducer 144 has been placed at the level of the reference point 130. Accordingly, an operative pressure reading should not be taken until the pressure transducer 144 has been placed at the level of the reference point 130, as determined from an indication by the level device 124 that it is level when the free ends 128 and 140 of the hinged level 104 are proximate to the reference point 130 and the pressure transducer 144.

At step 732, a determination may be made as to whether the patient has moved, the height or elevation of the patient 136 has changed, or whether any other event has occurred indicating that the level of the pressure transducer 144 relative to the reference point 130 should be checked. For example, in addition to movement of the patient 136, the level of the pressure transducer 144 relative to the reference point 130 should be checked if there has been an inadvertent change in the level of the pressure transducer 144, or if some predetermined period of time since the level was last checked has elapsed. If the position of the patient has changed or some other event indicating that the level should be rechecked has occurred, the process may return to step 712. At step 736, a determination may be made as to whether monitoring of the patient 136 should be continued. If monitoring should be continued, the process can return to step 712 to prepare for another reading. If the level process should be discontinued, that process may end.

In accordance with embodiments of the present invention, the elongate members 108 and 112 are formed from a rigid material. For example, and without limitation, the elongate members 108 and 112 can be formed from anodized aluminum, plastic, stainless steel, or copper. Markings 216, if provided, can be etched and/or printed. The hinge 116 can comprise a riveted element, a low profile swaged hinge, a plain bearing, a screw type fastener, or other mechanical interconnection that allows the elongate members 108 and 112 to pivot about a pivot axis 120, such that the elongate members 108 and 112 can be moved relative to one another in a first plane. In an exemplary embodiment, each elongate member 108 and 112 is about 20 inches long, 1.5 inches wide, and 0.09 inches thick.

Although certain exemplary embodiments have been illustrated as including a single level device 124, additional level devices 124 can be included. For example, a plurality of spirit levels, oriented at different angles from one another, could be disposed along the first elongate member 108. In addition, although fluid levels have been discussed and shown in exemplary embodiments, other embodiments of the disclosed invention can incorporate a level device 124 comprising an electronic level device, such as a laser level, or a series of accelerometers or inclinometers and a suitable control and output device.

The foregoing discussion of the invention has been presented for purposes of illustration and description. Further, the description is not intended to limit the invention to the form disclosed herein. Consequently, variations and modifications commensurate with the above teachings, within the skill or knowledge of the relevant art, are within the scope of the present invention. The embodiments described hereinabove are further intended to explain the best mode presently known of practicing the invention and to enable others skilled in the art to utilize the invention in such or in other embodiments and with various modifications required by the particular application or use of the invention. It is intended that the appended claims be construed to include alternative embodiments to the extent permitted by the prior art.

What is claimed is:

1. A hinged level device, comprising:
a first elongate member;
a second elongate member;
a hinge, wherein the hinge interconnects the first elongate member to the second elongate member, wherein the hinge defines a pivot axis, wherein the hinge permits the first elongate member and the second elongate member to be pivoted relative to one another in a first plane, and wherein the first plane is perpendicular to the pivot axis;
a level device interconnected to the first elongate member, wherein the level device has a reference plane that is parallel to the first plane.

2. The device of claim 1, wherein the level device includes a bubble level.

3. The device of claim 1, wherein the first and second elongate members are rigid.

4. The device of claim 3, wherein the first and second elongate members each include a first end and a second end, and wherein the second end of each of the first and second elongate members is proximate to the hinge.

5. The device of claim 4, wherein a distance between the first end of the first elongate member and the first end of the second elongate member can be varied by pivoting one of the elongate members relative to the other one of the elongate members about the pivot axis.

6. The device of claim 4, wherein the first end of the first elongate member is at about the same elevation as the first end of the second elongate member when the level device indicates that the hinged level device is level.

7. The device of claim 4, wherein the first elongate member includes a notch at the second end, and wherein the second elongate member includes indicia at the second end that is selectively revealed by the notch in the first elongate member as an angle between the first and second elongate members is changed by pivoting the first and second elongate members relative to one another about the pivot axis to provide an indication of a measure of the angle between the first and second elongate members.

8. The device of claim 1, wherein the hinge includes:
a pivot member fixed to the first elongate member centered on the pivot axis;
a hole in the second elongate member centered on the pivot axis that receives the axle member;
a fastener interconnected to the axle member on a side of the second elongate member opposite the first elongate member.

9. The device of claim 1, wherein the first and second elongate members each include first and second planar side surfaces that lie in planes that are parallel to a reference plane of the level device.

10. The device of claim 1, wherein a plurality of level devices are included in the device.

11. A method for leveling a reference point and a pressure transducer, comprising:
placing a free end of a first elongate member proximate to a reference point on a patient;
placing a free end of a second elongate member proximate to a pressure transducer, wherein the first elongate member is interconnected to the second elongate member by a hinge, wherein placing a free end of a second elongate member proximate to a pressure transducer includes changing an angle between the first and second elongate members by pivoting one of a first one of the first and second elongate members relative to a second one of the first and second elongate members about the hinge;
while the free end of the first elongate member is proximate to the reference point and while the second elongate member is proximate to the pressure transducer, referencing a level device interconnected to one of the first elongate member or the second elongate member to determine whether the first and second elongate members are level.

12. The method of claim 11, further comprising:
in response to determining that the first and second elongate members are not level, adjusting the elevation of the pressure transducer.

13. The method of claim 12, further comprising:
after adjusting the elevation of the pressure transducer:
placing the free end of the first elongate member proximate to the reference point on the patient;
placing the free end of the second elongate member proximate to the pressure transducer;
while the free end of the first elongate member is proximate to the reference point and while the second elongate member is proximate to the pressure transducer, referencing the level device interconnected to one of the first elongate member or the second elongate member to determine whether the first and second elongate members are level.

14. The method of claim 11, further comprising:
in response to determining that the first and second elongate members are level, reading a signal from the pressure transducer.

15. The method of claim 14, further comprising:
using the signal from the pressure transducer to assess a condition of the patient.

16. A hinged level, comprising:
a first elongate member;
a pivot member fixed to the first elongate member;
a second elongate member, including a hole that receives the pivot member;
a fastener interconnected to the pivot member, wherein the second elongate member is held between the fastener and the first elongate member, and wherein an angle between the first elongate member and the second elongate member can be changed within a first plane by pivoting the first elongate member relative to the second elongate member about the pivot member;
a level device interconnected to one of the first elongate member or the second elongate member, wherein a reference plane of the level device is parallel to the first plane.

17. The hinged level of claim 16, wherein the level device is a bubble level.

18. The hinged level of claim 16, wherein the level device is interconnected to the first elongate member.

19. The hinged level of claim 16, further comprising:
a notch at a second end of the first elongate member;
a set of indicia on the second elongate member, wherein a different element included in the set of indicia is revealed by the notch at different relative angles of the first elongate member and the second elongate member.

20. The hinged level of claim 16, wherein the free ends of the elongate members are within half an inch of the first plane.
